(19) United States
(12) Patent Application Publication
(10) Pub. No.: US 2007/0286880 A1
(43) Pub. Date: Dec. 13, 2007

(54) Inoculated spongiform scaffold for transplantation and tissue regeneration

(76) Inventors: Andrey Vasiliev, Moscow (RU); Ayvar Faizulin, Moscow (RU); Ivan Kiseliov, Moscow (RU); Olga Rocovaya, Reutov (RU); Nikolai Tankovich, San Diego, CA (US); Alexander Kharazi, San Diego, CA (US)

Correspondence Address: STEMEDICA CELL TECHNOLOGIES, INC, 5375 MIRA SORRENTO PLACE, SUITE 100, SAN DIEGO, CA, 92121, US

(21) Appl. No.: 11/450,625

(22) Filed: Jun. 8, 2006

(51) Int. Cl. A61K 9/00; C12N 5/10; C12Q 1/02

(52) U.S. Cl. 424/422; 424/93.21; 435/29; 435/395

(57) ABSTRACT

A spongiform scaffold which comprises epithelial stem cells, and free of mesenchymal cells. A spongiform scaffold comprising precursor keratinocytes for use in a method of transplantion of the scaffold to an epithelial cell target site in a recipient, resulting in growth of said epithelial stem cells and the ingrowth of cells from the body of said recipient to restore tissue. A version of the scaffold is formed from collagen, and in particular, Spongostan™.

### FIELD OF THE INVENTION

The present invention generally relates to tissue engineering and specifically relates to scaffolding for cell and tissue culture. In particular, the present invention relates to an epithelial-cell inoculated sponge scaffold for use in cell transplantation and/or organ reconstruction. In particular, the invention relates to living skin equivalents which combine epidermal-derived keratinocyte cells and a spongiform scaffold for transplantation. The invention further relates to methods for using these compositions as a treatment for epithelial defects including laryngeal defects, urogenital defects and burns.

### BACKGROUND

Tissue engineering offers a novel route for repairing damaged or diseased tissues by incorporating the patients' own healthy cells or donated cells into temporary housings or scaffolds. The structure and properties of the scaffold are critical to ensure normal cell behavior and performance of the cultivated tissue.

This new tissue engineering approach is just beginning to be commercially exploited in products such as skin substitutes. Once the technology has been sufficiently developed, cells grown on a porous scaffold will be used to repair tissues within the human body.

In order to study the therapeutic effects of skin substitutes, a few investigators have explored the use of three-dimensional substrates such as collagen gel (Douglas et al., (1980) In Vitro 16:306-312; Yang et al., (1979) Proc. Natl. Acad. Sci. 76:3401; Yang et al. (1980) Proc. Natl. Acad. Sci. 77:2088-2092; Yang et al., (1981) Cancer Res. 41:1021-1027); cellulose sponge, alone (Leighton et al., (1951) J. Natl Cancer Inst. 12:545-561) or collagen coated (Leighton et al., (1968) Cancer Res. 28:286-296); and a gelatin sponge commercially known as Gelfoam (Sorour et al., (1975) J. Neurosurg. 43:742-749).

A wide variety of medical conditions exist that can be improved or corrected using a three dimensional tissue scaffolding that serves as a support system for cells intended to grow and replace missing and/or damaged tissue. These medical conditions range from acute trauma caused by car accidents, to degenerative disease in which tissue structure and function are compromised or lost. The challenge has been to identify and develop systems that will replace or enable the body to regenerate lost or damaged tissue.

Skin

The skin consists of two types of tissue which are: (1) the stroma or dermis which includes fibroblasts that are loosely dispersed within a high density collagen matrix comprising nerves, blood vessels and fat cells; and (2) the epidermis which includes an epidermal basal layer of tightly packed, actively proliferating immature epithelial cells.

As the cells of the basal layer replicate, some remain in the basal layer while, others migrate outward, increase in size and eventually differentiate into keratinocytes which are resistant to detergents and reducing agents. In humans, cells born in the basal layer take about 2 weeks to reach the outer layer of the skin where the cells die and are eventually shed.

The skin contains various structures including hair follicles, sebaceous glands and sweat glands. Hair follicles are formed from differentiating keratinocytes that densely line invaginations of the epidermis. The open-ended vesicles that form from such invaginations collect and concentrate the secreted keratin resulting in a hair filament. Alternatively, epidermal cells lining an invagination may secrete fluids (sweat gland) or sebum (sebaceous gland). The regulation of formation and proliferation of these structures is unknown. The constant renewal of healthy skin is accomplished by a balanced process in which new cells are being produced and aged cells die.

The health and integrity of skin may be compromised by congenital or acquired pathological conditions for which normal skin regeneration and repair processes may be inadequate. Without limitation, these conditions include burns, wounds, ulcers, infections, and/or congenital abnormalities. Patients who are burned over a large surface area often require immediate and extensive skin replacement. Less life-threatening but chronic skin conditions, as occur in venous stasis ulcers, diabetic ulcers, or decubitus ulcers as three examples, may progress to more severe conditions if left untreated, particularly since patients with these conditions have an underlying pathology. Reduction of morbidity and mortality in such patients depends upon timely and effective restoration of the structure and function of skin.

Below the epidermis is a layer of cells and connective tissue called the dermis. This layer comprises mesenchymal cells, which includes fibroblast cells and cells of blood and lymph vessels. Hair follicles, sebaceous glands, and sweat glands extend from the dermis to the surface of the skin. These glands and follicles are lined by epithelial cells.

Cultured Skin

A cultured skin is a comparatively well-developed example in the field of tissue models and artificial organs. A cultured skin includes: skin prepared by culturing human fibroblasts in collagen gel, followed by inoculating and culturing human keratinocytes on the gel when the gel is shrunk (U.S. Pat. No. 4,485,096); skin prepared by inoculating and culturing human fibroblast on nylon mesh, followed by inoculating and culturing human keratinocyte thereon when pores of the mesh are filled up with secreted materials from fibroblasts (Slivka, S. R., L. Landeen, Zimber, M., G. K. Naughton and R. L. Bartel, J. Invest. Dermatol., 96: 544A, 1991); and skin prepared by inoculating and culturing human fibroblasta in a collagen sponge, followed by laminating collagen gel or film inoculating and culturing human keratinocyte thereon (J. Jpn. P. R. S., 10, 165-180 (1990) and Japanese Examined Patent Publication No. 47043/1995).

The most important problem in producing tissue models is reconstructing a three-dimensional structure of tissues or organs as quickly as possible. For example, a skin mainly comprises keratinocytes in the epidermis, fibroblasts in the dermis and inter-cellular substances such as collagen, which are not existent in a mixed form. A skin comprises a dermis layer formed by three-dimensional proliferation of fibroblasts in a collagen fiber matrix, and an epidermal layer formed thereon by repeatedly laminating keratinocytes in a complex process wherein basal layer cells differentiate into a corneous layer.

The use of fibroblasts also presents a challenge to the production of therapeutic tissue models. Although fibroblasts provide growth factors and other cell-to-cell contacts that facilitate cell division, their proliferation may outpace epidermal cell division resulting in a culture that is overgrown with fibroblasts. This is clearly undesirable as therapies aimed at the regeneration of epidermal tissues must be carried out using carriers rich in epidermal cells. One means of preventing the overgrowth of fibroblast involves plating the epidermal cells with irradiated 3T3 (mouse) cells. Rheinwald and Green, Cell, 6, 331-334, November 1975. However this technique requires the presence of dermal components which is undesirable in therapeutic applications.

Materials have been manufactured for use in permanent skin repair. These materials contain different components that replace or simulate the components and functions of the dermis and/or epidermis. Examples of these materials include the following: EpiCel™, which lacks a dermal component and uses the patient's own cultured keratinocytes; Integra™, which uses a collagen-glycosaminoglycan (GAG) matrix to provide an acellular dermal component and uses a thin epidermal autograft; AlloDerm™, which uses a dermal matrix and a thin epidermal autograft; DermaGraft™, which uses a polyglycolic acid/polylactic acid (PGA/PLA) matrix and allogeneic human fibroblasts for the dermis; Hyaff/LaserSkin™, which uses hyaluran and fibroblasts for the dermis, and hyaluran and the patient's own keratinocytes for the epidermis; and PolyActive™, which uses polyethylene oxide/polybutylthalate (PEO/PBT) and the patient's own fibroblasts for the dermis, and the patient's cultured keratinocytes for the epidermis.

Materials to either temporarily cover wounds, or to stimulate permanent skin repair processes, include: ApliGraft™, which uses collagen gel and allogeneic fibroblasts for the dermis, and cultured allogeneic keratinocytes for the epidermis; Comp Cult Skin™ or OrCel™, which uses collagen and allogeneic fibroblasts for the dermis, and cultured allogeneic keratinocytes for the epidermis; and TransCyte™, which uses allogeneic fibroblasts for the dermis and a synthetic material, BioBrane™, for the epidermis.

Yannas et al. in U.S. Pat. No. 4,458,678 disclose a method for preparing a fibrous lattice and seeding it with viable cells. The lattice is prepared by pouring an aqueous slurry of collagen and glycosaminoglycan into an open metal tray or pan.

U.S. Pat. No. 5,976,878 discloses a device which has been used for permanent skin replacement. This device is applied surgically in a single procedure, and contains a layer of cultured epidermal cells, a synthetic dermal membrane component, and a substantially nonporous synthetic lamination layer on one surface of the dermal membrane component. The synthetic dermal membrane component is formed from collagen, or collagen and a mucopolysaccharide compound, and is laminated with the same collagen or collagen and mucopolysaccharide compound-containing solution containing a volatile cryoprotectant. The substantially nonporous lamination layer may be located between the dermal component and the layer of cultured epidermal cells, promoting localization of epidermal cells on the surface of the dermal component and movement of nutrients to the cells of the cellular epidermal component.

Recently, acellular artificial skins or cell-based bioartificial skins have been developed and marketed. Examples include acellular artificial skins, such as an acellular collagen-glycosaminoglycan matrix bonded to a thin silicone membrane (INTEGRA™, Interga LifeSciences Co.) and dehydrorothermally cross-linked composites of fibrillar and denatured collagens (Terudermis.™., Terumo Co.), are now commercially available. However, such products are very expensive because they incorporate biomaterials such as collagen and thus, have difficulty in clinical trials on broad wound sites, e.g., burns.

Advanced Tissue Sciences, Inc. (La Jolla, Calif.) developed a skin replacement product composed of a thin biodegradable mesh framework onto which human dermal fibroblasts are seeded, for use in treating diabetic foot ulcers (Dermagraft-TC™). Other skin replacements include an epidermal cell sheet for partial-thickness wounds (Acticel™, Biosurface Technology, Inc.), composite grafts of cultured keratinocytes and fibroblasts on a collagen glycosaminoglycan matrix (Apligraft™, Organogenesis, Inc.) and a skin replacement product derived from human cadaver skin (Alloderm™, Lifecell).

Skin grafting of denuded areas, granulating wounds and burns still present major healing problems despite advances in grafting techniques. Split thickness autografts and epidermal autografts (cultured autogenic keratinocytes) have been used with variable success.

Conventional tissue models and artificial organs are limited by the lack of a three-dimensional structure. Despite progress in the development of cultured skins, conventional tissues and organs take more than one month to prepare from the inoculation of cells, to completion of skin reconstruction. Also, keratinocyte laminates are slow to differentiate when compared to actual human skin.

Thus, there is a need for the development of living skin equivalent grafts which comprise proliferating and differentiating cells that can be easily prepared and maintained in sufficient quantities to enable treatment of skin wounds.

In developing a living skin equivalent it is desirable that it comprise at least some or all of the following features: it should enable rapid and sustained adherance to the wound surface, it should be tissue comparable, it should have an inner surface in contact with the wound surface that promotes the ingrowth of fibrovascular tissue, and/or it should provide protection from infection and prevention of fluid loss.

### SUMMARY

The invention provides a spongiform scaffold which comprises epithelial stem cells. The combination of the scaffold and epithelial cells is fee of mesenchymal cells. An embodiment of the spongiform scaffold involves epithelial stem cells which are precursor keratinocytes. The spongiform scaffold is used in a method of the invention which involves transplantion of the scaffold to a target site in a recipient, the scaffold permits the growth of said epithelial stem cells and the ingrowth of cells from the body of said recipient. A version of the scaffold is formed from collagen, and in particular, Spongostan™.

Another aspect of the invention involves a method for generating or regenerating tissue in a subject. The method involves delivering an epithelial stem cell-inoculated spongiform scaffold free of mesenchymal cells to a epithelial defect target site in a recipient. After delivery, the scaffold permits the epithelial stem cells inoculated on the spongiform scaffold to differentiate thereby producing epithelial tissue at the target site.

Included in the invention is a method of making an inoculated spongiform scaffold for treating an epithelial defect in a recipient. The method involve inoculating a spongiform scaffold with a sufficient number of epithelial stem cells in an inoculum. Preferably, the inoculum includes enough cells to restore the epithelium at said epithelial defect.

In certain embodiments, the spongiform scaffold and methods of using it are adapted for treating skin defects, and in others urological defects, in particuar hypospadias.

### DETAILED DESCRIPTION OF THE INVENTION

The present invention relates to a spongiform scaffold having a structure suitable for use as a biologically active skin equivalent, and processes for preparing and using said construct. Prior to actual use as a biologically active skin equivalent, the construct, which is free of mesenchymal cells, is inoculated with appropriate epithelial stem cells which are free of mesenchymal cells.

The present invention further relates to a biocompatible material comprising a spongiform scaffold that is inoculated with epithelial stem cells, wherein said material is free of mesenchymal cells and capable of regenerating epithelial tissue when implanted in a subject. The biocompatible material of the invention may comprise one or more epithelial stem cell lines.

The essence of the scaffold of the present invention is a porous spongiform scaffold containing an inoculum of epithelial stem cells free of mesenchymal cells. In some embodiments, the spongiform scaffold is inoculated with precursor keratinocyte cells.

Certain embodiments of the scaffolds further comprise growth promoters, as detailed below. The inoculated scaffold may alternatively be cryogenically stored for later use in tissue reconstruction. Other aspects of the invention relate to methods of using the inventive scaffold for treating epithelial defects including laryngeal and urogenital defects.

In one embodiment, the spongiform scaffold of the invention is employed for wound healing. Repair of skin lesions is known to be a highly complex process that includes primary epithelial cell migration as well as replication of epidermal cells in response to molecular signals from underlying connective tissue. Inoculated spongiform scaffolds are described herein as a model for wound healing. Moreover, the inventive inoculated spongiform scaffolds are used to treat burn patients. Several centers in the United States and Europe have utilized cultured human keratinocyte allografts and autografts to permanently cover the wounds of burns and chronic ulcers (Eisinger et al., (1980) Surgery 88:287-293; Green et al., (1979) Proc. Natl. Acad. Sci. USA 76:5665-5668; Cuono et al., (1987) Plast. Reconstr. Surg. 80:626-635). These methods are often unsuccessful and recent studies have indicated that blistering and/or skin fragility in the healed grafts may exist because of an abnormality in one or more connective tissue components formed under the transplanted epidermal layer (Woodley et al., (1988) JAMA 6:2566-2571). In some aspects of the invention, the inoculated spongiform scaffolds provide a skin equivalent for treating burns in a recipient.

Any epithelial tissue can be treated with the inventive scaffolding described herein. Without limitation, these tissues include the skin, gastrointestinal epithelium, respiratory epithelium, and urinary tissues. In particular, the inventive scaffolding has application in the treatment of laryngeal and urethral defects.

### DEFINITIONS

The term “tissue” as used herein refers to an aggregation of similarly specialized cells united in the performance of a particular function. Tissue is intended to encompass all types of biological tissue including both hard and soft tissue. A “tissue” is a collection or aggregation of particular cells embedded within its natural matrix, wherein the natural matrix is produced by the particular living cells. The term may also refer to ex vivo aggregations of similarly specialized cells which are expanded in vitro such as in artificial organs.

The term “epithelial cell” as used herein refers to any cell that is found in an epithelial tissue. The term includes epithelial stem cells, as well as terminal cells including keratinocytes.

The term “epithelial stem cell” as used herein refers to a cell that is capable of dividing and differentiation into a mature epithelial cell. Precursor keratinocytes are one example of an epithelial stem cell. Large proportions of epithelial stem cells occupy the basal layer of the epidermis, as well as neonatal foreskin (see e.g. Alonso, L “Stem cells of the skin epithelium” PNAS (2003) 100; supp. 1: 11830-11835; and Tumbar, T. “Essentials of Stem Cell Biology” (2006), the disclosures of which are incorporated herein by reference).

The terms “precursor cell,” “tissue precursor cell” and “progenitor cell” are used interchangeably herein and refer to lineage-committed cells that divide and differentiate to form new, specialized tissue(s). As used herein, the terms “iprecursor cell” and “progenitor cell” are also intended to encompass a cell which is sometimes referred to in the art as a “stem cell” in that like precursor and progenitor cells, stem cells divide and form new phenotypically different tissues. It should be understood that an “epidermal progenitor cell” is used interchangebly with the terms “progenitor keratinocyte” and “precursor keratinocyte” to denote regenerative cells of the epidermis. Epidermal progenitor cells as disclosed herein are regenerative and differentiate into terminal keratinocytes. The precursor keratinocytes of the present invention are found in epithelial tissues including, but not limited to, the outer root hair sheath, the corneal limbus, the hair bulge and neonatal foreskin.

The term “gel” as used herein refers to a colloidal material having the consistency of a viscous semi-ridgid sol. The term “gel” also refers to the act of forming such a colloidal material or any similar semi-solid material.

The term “gelatin” as used herein refers to a gel that is obtained by the partial hydrolysis of collagen. Without limitation, the gelatins described herein may be derived from the skin, white connective tissue, and/or the bones of animals. Gelatins may be used to produce the bioabsorbable spongiform scaffolds disclosed herein.

The term “gelatiniferous” as used herein refers to the ability to produce gelatin.

The term “gelatinize” as used herein refers to the conversion of a substance into a gel-like consistency.

The term “gelatinoid” or “gelatinous” are used interchangeably herein and refer to a gelatin or jelly-like consististency.

The term “gelation” as used herein refers to refers to the conversion of a sol into a gel.

The terms “Spongostan”™ [USP] and “Gelfoam”™ [USP] as used herein refer to commercial absorbable spongiform scaffolds respectively produced respectively by Johnson and Johnson and Upjohn. These sponges are water-insoluble, off-white, nonelastic, porous, pliable products prepared from purified pork Skin Gelatin [USP] granules and water and are able to absorb and hold within their interstices, many times its weight of blood and other fluids.

The term “sponge” and “spongiform” are used interchangeably herein and refer to any porous, biocompatible material capable of supporting the growth and implantation of the cells disclosed herein. Examples of sponges include, without limitation, gauzes and other porous materials such as foams. The term “sponge” further includes any structure having open spaces therein and which supports the migration and growth of human fibroblasts.

The term “absorbable gelatin sponge” (“AGS”) [USP] as used herein refers to a sterile, absorbable, water-insoluble gelatin-based sponge that is commonly used as a local hemostatic. The AGS can be of any desired shape including, but not limited to planar shapes, sac-like shapes, tubular shapes, and combinations thereof. The shape of the AGS is chosen to best correct any physical defect in the patient. Spongostan™ and Gelfoam™ are examples of an absorable gelatin sponge that are commercially available from Johnson and Johnson and Upjohn.

The term “spongiform” means resembling a sponge such as an absorbable gelatin sponge.

The term “spongi-” is a combining form meaning like a sponge, or denoting a relationship to a sponge.

The term “spongy” refers to a spongelike consistency or texture.

The term “scaffold” as used herein refers to a three-dimensional spongiform supporting structure for growing cells and tissues. Examples of scaffolds include, but are not limited to Spongostan™ and Gelfoam™.

The term “substrate” refers to any substance that can be used for the culture and therapeutic application of the cells disclosed herein. Without limitation, the term includes spongiform porous scaffolds made from a biocompatible spongiform material.

The term “support structure,” or “supporting structure,” as used herein refers to a reinforcing material that is associated with the spongiform scaffold. Supporting structures may overlay, or be embedded within the spongiform scaffold. These structures increase the strength and/or rigidity of the spongiform scaffold making it resistant to forces such as tearing and crushing. Supporting structures for use with the invention may be manufactured from any biocompatible material including biodegradable and non-biodegradable materials. Examples of supporting structures include, but are not limited to, catheters, tubes, stents, posts, hooks, bands, coils and linear arrangements of fibers such as meshes and fabrics.

The term “reinforce,” or “reinforcing,” as used herein refers to the placement of a support structure on, next to, surrounding or within a spongiform scaffold.

The term “biodegradable” as used herein refers to a material that contains bonds that may be cleaved under physiological conditions, including enzymatic or hydrolytic scission of chemical bonds. Non-biodegradable materials do not undergo this form of degradation and are not absorbed when placed in the body of an animal.

The term “biocompatible” is used herein to describe a material that does not cause any injury, toxic reaction or immunological reaction with a living tissue. Biologically compatible materials are used for the in vitro culture and/or implantation of the cells disclosed herein.

The terms “restore,” “restoration” and “correct” are used interchangeably herein and refer to the regrowth, augmentation, supplementation, and/or replacement of a defective tissue with a new and preferentially functional tissue. The terms include the complete and partial restoration of a defective tissue. Defective tissue is completely replaced if it is no longer present following the administration of the inventive composition. Partial restoration exists where defective tissue remains after the inventive composition is administered.

The term “irregular shape” as used herein refers to shapes that are assymetrical.

The terms “elastic” and “inelastic” refer to the resilience of a material. A material is elastic if it can be deformed without breaking, shattering, shearing or otherwise compromising the integrity of the material. Materials which do not have this property are inelastic.

The term “differentiate” as used herein refers to the process whereby an unspecialized cell acquires the features of a specialized cell. Differentiated cells have distinctive phenotypic characteristics and may perform specific functions.

The term “cell lineage” as used herein refers to a developmental pathway which a cell commits to as it differentiates from a less differentiated cell. Examples of embryonic cell lineages include ectodermal, endodermal and mesodermal germ lineages. Cell lineages also include adult cell pathways that characterize the development of specific terminal cells.

The term “cell line” as used herein refers to a population of cells cultured in vitro that are descended through one or more generations (and possibly cultures) from a single primary culture. The cells of a cell line share common characteristics.

The term “biomaterial” as used herein refers to a natural or synthetic biocompatible material that is suitable for introduction into living tissue, especially in connection with a medical device. A natural biomaterial is a material that is made by a living system. Synthetic biomaterials are materials which are not made by a living system. The biomaterials disclosed herein may be a combination of natural and synthetic biocompatible materials.

The term “biological activity” as used herein refers to the effect an agent has on a cell or population of cells. Effects that fall within the scope of this term include, but are not limited to, cytotoxicity, mutagenicity, proliferation, permeability, apoptosis, gene regulation, protein expression, and differentiation. Drug efficacy, or the desired effect of a test agent, is also encompassed by the term “biological activity.”

The term “hydrogel” as used herein refers to a substance that is formed when an organic polymer (natural or synthetic) is set or solidified to create a three-dimensional open-lattice structure that entraps molecules of water or other solution to form a gel. The solidification can occur, e.g., by aggregation, coagulation, hydrophobic interactions, or cross-linking. Hydrogel dressings are complex lattices in which the dispersion medium is trapped rather like water in a molecular sponge. Available hydrogels are typically insoluble polymers with hydrophilic sites, which interact with aqueous solutions, absorbing and retaining significant volumes of fluid. Hydrogel dressings are non-adherent and have a higher water content. Hydrogels have been reported to increase epidermal healing. Hydrogels progressively decrease their viscosity as they absorb fluid. In liquefying, hydrogels conform to the shape of the wound and their removal is untraumatic.

The term “hydrogel-cell composition” as used herein refers to a suspension of a hydrogel containing selected tissue precursor cells. These cells can be isolated directly from a tissue source or can be obtained from a cell culture.

The term “polymer” as used herein, means any molecule consisting of two or more molecular units.

The term “explant” as used herein refers to a collection of cells from an organ, taken from the body of an individual and grown in an artificial medium. When referring to explants from an organ having both stromal and epithelial components, the term generally refers to explants which contain both components in a single explant from that organ.

The term “organ” as used herein refers to two or more adjacent layers of tissue which maintain some form of cell-cell and/or cell-matrix interaction to generate a microarchitecture.

The term “stroma” as used herein refers to the supporting tissue or supporting matrix of an organ. Stromal cells are mesenchymal in origin. Fibroblasts are one example of a stromal cell.

The terms “mesenchymal,” “mesenchyme” and “mesodermal” are used interchangeably herein to refer to a cell that is derived from the mesoderm germ layer. Mesenchymal cells include connective tissue cells such as fibroblasts.

When used to refer to a population of cells, the term “isolated” includes a population of cells which results from the proliferation of cells in the micro-organ culture of the invention, or to a population of cells which results from the proliferation of cells isolated from a tissue or from a micro-organ culture.

The term “clone” and “clonal cells” are used interchangeably herein and refer to a cell that is produced by the expansion of a single, isolated cell. The term “clonal population” in reference to the cells of the invention shall mean a population of cells that is derived from a clone. A cell line may be derived from a clone and is an example of a clonal population.

When referring to a mass of tissue, the term “isolated” as used herein refers to an explant which has been separated from its natural environment in an organism. This term includes gross physical separation from the explant's natural environment, e.g., removal from the donor animals, e.g., a mammal such as a human. For example, the term “isolated” refers to a population of cells which is an explant, is cultured as part of an explant, or is transplanted in the form of an explant.

The term “ectoderm” as used herein refers to the outermost of the three primitive germ layers of the embryo which give rise to epithelial tissues, for example epidermis and glands in the skin, the nervous system, external sense organs and mucous membrane of the mouth, anus, urethra and larynx. The term “ectodermal” also refers to cells possessing the characteristics of this embryonic germ layer. One skilled in the art will appreciate that such cells need not be derived from embryonic tissues in that any cell that is capable of differentiating into cells that belong to the ectodermal lineage will be called an “ectodermal stem cell.” The skilled artisan will appreciate that any source of multipotent ectodermal stem cells may be used. Such sources include the in vitro differentiation of embryonic stem cells into lineage-committed ectodermal cells as disclosed in U.S. Patent Application No. 2002/0151056 A1, the disclosure of which is incorporated herein by reference.

The terms “epithelia” and “epithelium” as used herein refer to the cellular covering of internal and external body surfaces (cutaneous, mucous and serous), including the glands and other structures derived therefrom, e.g., corneal, esophageal, laryngeal, epidermal, hair follicle and urethral epithelial cells. Other exemplary epithelial tissues include: olfactory epithelium, which is the pseudostratified epithelium lining the olfactory region of the nasal cavity, and containing the receptors for the sense of smell; glandular epithelium, which refers to epithelium composed of secreting cells; squamous epithelium, which refers to epithelium composed of flattened plate-like cells. The epidermis is composed of squamous epithelium cells and provides one example of an epithelial tissue. The term epithelium can also refer to transitional epithelium, which is that characteristically found lining hollow organs, such as the larynx and urethra, that are subject to great mechanical change due to contraction and distention, e.g. tissue which represents a transition between stratified squamous and columnar epithelium. Epithelia originate from epithelial stem cells.

The term “epithelial defect” as used herein refers to any disease, condition, malformation, infection or trauma that compromises the appearance and/or function of an epithelial tissue. The term includes, without limitation, diabetic ulcers, urogenital defects (e.g. hypospadia), acne, and laryngeal abnormalities. Epithelial defect also includes mechanical, chemical and/or thermal injuries including burns, abrasions and surgical wounds. Epithelial defect further includes microinjuries to the epithelium which are induced in aesthetic procedures such as a lasering, mechanical dermabrasions, electromagnetic and ionizing radiation of the skin and chemical peeling. Moreover, the term “epithelial defect” includes any epithelial condition that can be treated by the replacement, augmentation or regeneration of the defective epithelial tissue. An epithelial defect is improved if the negative effects or malformed appearance of the epithelial defect is reduced or eliminated.

The term “target site” as used herein refers to the location of an epithelial defect in a subject. The term includes the space occupied by the epithelial defect, as well as the defect's periphery. The inventive composition is adapted for placement on a target site. Methods of the invention involve placing the inventive composition at a target site to correct an epithelial defect.

The terms “subject” and “recipient” as used herein refer to an individual that receives, or is intended to receive, the inventive composition using the methods of the invention. The terms include any animal having epithelial tissues including mammals such as humans and primates. The term “xenogeneic subject” refers to a subject that is a different species than the subject that receives, or is intended to receive, a biological material from the xenogeneic subject. An “allogeneic subject” is a subject into which cells of the same species are introduced or are to be introduced. Donor subjects are subjects which provide the cells, tissues, or organs, which are to be placed in culture and/or transplanted into a recipient. Recipients of a donated material can be either a xenogeneic or an allogeneic recipient. Donor subjects can also provide cells, tissues, or organs for reintroduction into themselves, i.e. for autologous transplantation. In cases of autologous transplantion, the recipient and donor are the same individual.

The terms “administer,” “treat,” “deliver,” “provide,” “deliver,” “transplant” and “introduce” are used interchangeably herein and refer to the application of the inventive composition to a subject under conditions that results in the delivery of epithelial stem cells to a desired location in the subject where at least a portion of the cells remain viable. The inventive composition may be administered by placing it within, or on the surface of, a subject's body at a target site of an epithelial defect. This placement results in localization of epithelial stem cells to a desired site. The cell populations can be administered to a subject by any appropriate route

The term “substantially fit” as used herein refers to the shaping of the spongiform scaffold to conform to an epithelial defect. A shaped spongiform scaffold “substantially fits” an epithelial defect if a majority of at least one surface of the spongiform scaffold is in contact with the surface of the epithelial defect.

The term “epithelialization” as used herein refers to healing by the growth of epithelial tissue over a surface.

The term “skin” as used herein refers to the outer protective covering of the body, consisting of the dermis and the epidermis, and is understood to include sweat and sebaceous glands, as well as hair follicle structures. Throughout the present application, the adjective “cutaneous” may be used, and should be understood to refer generally to attributes of the skin, as appropriate to the context in which they are used. The term “skin defect” as used herein refers to an epithelial defect in the epidermis.

The term “epidermis” as used herein refers to the outermost and nonvascular layer of the skin, derived from the embryonic ectoderm, and varying in thickness from 0.07-1.4 mm. On the palmar and plantar surfaces it comprises, from within outward, five layers: basal layer composed of columnar cells arranged perpendicularly; prickle-cell or spinous layer composed of flattened polyhedral cells with short processes or spines; granular layer composed of flattened granular cells; clear layer composed of several layers of clear, transparent cells in which the nuclei are indistinct or absent; and horny layer composed of flattened, cornified non-nucleated cells. In the epidermis of the general body surface, the clear layer is usually absent.

The “dermis” as used herein refers to the layer of the skin beneath the epidermis, consisting of a dense bed of vascular connective tissue, and containing the nerves and terminal organs of sensation. The hair roots, and sebaceous and sweat glands are structures of the epidermis which are deeply embedded in the dermis.

The term “micro-organ culture” as used herein refers to an isolated population of cells, e.g., an explant, having a microarchitecture of an organ or tissue from which the cells are isolated. That is, the isolated cells together form a three dimensional structure which simulates/retains the spatial interactions, e.g. cell-cell, cell-matrix and cell-stromal interactions, and the orientation of actual tissues and the intact organism from which the explant was derived. Accordingly, such interactions as between stromal and epithelial layers is preserved in the explanted tissue such that critical cell interactions provide, for example, autocrine and paracrine factors and other extracellular stimuli which maintain the biological function of the explant, and provide long term viability under conditions wherein adequate nutrient and waste transport occurs throughout the sample.

The term “signal,” or “cell signal” as used herein refers to an extracellular or intracellular molecule that cues the response of a cell to the behavior of other cells or objects in the environment (“Molecular Biology of the Cell” 4^{th} Ed. (2002) p. G:32).

The term “gland” as used herein refers to an aggregation of cells specialized to secrete or excrete materials not related to their ordinary metabolic needs. For example, “sebaceous glands” are holocrine glands in the corium that secrete an oily substance and sebum. The term “sweat glands” refers to glands that secrete sweat, situated in the corium or subcutaneous tissue, opening by a duct on the body surface. The ordinary or eccrinesweat glands are distributed over most of the body surface, and promote cooling by evaporation of the secretion; the apocrine sweat glands empty into the upper portion of a hair follicle instead of directly onto the skin, and are found only in certain body areas, as around the anus and in the axilla.

The terms “hair” and “pilus” are used interchangeably herein and refer to a threadlike structure, especially the specialized epidermal structure composed of keratin and developing from a papilla sunk in the corium, produced only by mammals and characteristic of that group of animals. The term also refers to the aggregate of such hairs. A “hair follicle” refers to one of the tubular-invaginations of the epidermis enclosing the hairs, and from which the hairs grow; and “hair follicle epithelial cells” refers to epithelial cells which are surrounded by the dermis in the hair follicle, e.g., stem cells, outer root sheath cells, matrix cells, and inner root sheath cells. Such cells may be normal non-malignant cells, or transformed/immortalized cells.

The terms “proliferating” and “proliferation” as used herein refer to cells undergoing mitosis.

The term “transformed cells” as used herein refers to cells which have been modified through genetic engineering manipulations to a state of unrestrained growth, i.e., they have acquired the ability to grow through an indefinite number of divisions in culture. Transformed cells may be characterized by such terms as neoplastic, anaplastic, immortalized and/or hyperplastic, with respect to their loss of growth control.

The term “genetically modified” and “genitically altered” are used interchangeably herein and refer to cells that contain and which may express one or more exogenous polynucleotide(s).

The term “immortalized cells” as used herein refers to cells which have been altered via chemical and/or recombinant means such that the cells have the ability to grow through an indefinite number of divisions in culture.

The term “epidermal equivalent” as used herein means an in vitro generated organotypic tissue culture resembling in its histological structure the natural epidermis especially concerning the stratification and development of the horny layer. A normal stratified epidermis consists of a basal layer of small cuboidal cells, several spinous layers of progressively flattened cells, a prominent granular layer and an orthokeratotic horny layer. All these layers can be detected in epidermal equivalents. Localization of those epidermal differentiation products that have been assayed by immunohistochemistry (e.g. keratins, involucrin, filaggrin, integrins) is similar to that found in normal epidermis.

The term “autologous” as used herein means: (i) that biological material to be transplanted is derived from the individual to be treated with epidermal equivalents; or (ii) that biological material added to tissue cultures comes from the donor of the cells for tissue culture. The term “autologous” is used to indicate that a biological material is genetically identical to, and/or derived from, a selected individual.

A “test agent” is any substance that is evaluated for its ability to diagnose, cure, mitigate, treat, or prevent disease in a subject, or is intended to alter the structure or function of the body of a subject. Test agents include, but are not limited to, chemical compounds, biologic agents, proteins, peptides, nucleic acids, lipids, polysaccharides, supplements, signals, diagnostic agents and immune modulators. In some aspects of the invention, test agents include electromagenetic and/or mechanical forces.

The term “electromagnetic force” as used herein refers to a force that results from kinetic electrical energy. Examples of electromagnetic forces, without limitation, include lasers, magnetic fields and electric current.

The term “homologous” as used herein means: (i) that biological material to be transplanted is derived from one or more individuals of the same species as the individual to be treated with epidermal equivalents; or (ii) that biological material added to tissue cultures comes from one or more individuals of the same species as the donor of cells for the tissue culture.

The term “organotypic culture” as used herein refers to a culture of cells under conditions that promote differentiation of the cells. Under conditions of organotypic culture, proliferation of the cells is slowed compared to culture under “proliferative” conditions such as primary culture conditions, and may be completely stopped.

The terms “inoculation” and “seeding” are used interchangeably herein and refer to the introduction of cells to a substrate such as a spongiform scaffold. Seeding cells at a “density sufficient to correct an epithelial defect” means the cells on the seeded substrate are large enough in number, per square unit area of scaffold, to restore the epithelial defect. The inoculation of a substrate may, or may not, involve the in vitro expansion of the cells in culture.

The term “inoculum” as used herein refers to the cells introduced or to be introduced to a spongiform scaffold. An inoculum may consist of cells from one or more cell lines.

The term “xenogeneic” as used herein is used to indicate that a donor biological material is derived from a different species than the recipient of the biological material.

### Epithelial Stem Cells

The inventive composition is seeded with epithelial stem cells. Epithelial stem cells are responsible for regenerating keratinocytes. The epithelial stem cells of the inventive composition are present in a variety of tissue compartments including the basal layer of the epidermis, the hair bulge, neonatal foreskin and the corneal limbus (Ghazizadeh, S. “Organization of stem cells and their progeny in human epidermis” J. Invest. Dermatol. (2005) 124(2):367-72; Watt F M. “Epidermal stem cells: markers, patterning and the control of stem cell fate” Philos. Trans. R. Soc. Lond. B. Biol. Sci. (1998) 353(1370):831-7; Ito, M. “Stem cells in the hair follicle bulge contribute to wound repair but not to homeostasis of epidermis” Nat. Med. (2005) 1(12):1351-1354; Ito, M. “Hair follicle stem cells in the lower bulge form the secondary germ, a biochemically distinct but functionally equivalent progenitor cell population, at the termination of catagen” Differentiation (2004) 72(9-10):548-557; Chee, K. Y. “Limbal stem cells: the search for a marker” Clin. Exper. Opthamol. (2006) 34(1):64-73; and Webb A “Location and phenotype of human adult keratinocyte stem cells of the skin” Differentiation (2004) 72(8):387-95).

The epithelial stem cells of the inventive composition may be derived from post-natal and prenatal tissues (see e.g. Zhou, J. X. “Enrichment and identification of human ‘fetal’ epidermal stem cells” Hum. Reprod. (2004) 19(4):968-74). Moreover, in the case of adult-derived epithelial stem cells, cells may be autologous or homologous in nature. Homologous epithelial stem cells are preferred since they provide a supply of cells that can be prepared in advance thereby eliminating the need for a patient to wait while their own autologous cells are expanded ex vivo. In the case of burn treatments, homologous preparations allow patients to be covered in a single procedure without the need for painful autografts which may become infected.

In another aspect of the inventive composition, the epithelial stem cells are autologous stem cells. In general, this embodiment relies on harvesting the patient's own epithelium-forming cells, expanding them ex vivo, and seeding the expanded cells on spongiform scaffolds for delivery according to the methods of the invention. By increasing the number of the patient's own epidermal stem cells and incorporating them directly into the inventive composition, a normal and fully-functional multilayer skin can be restored using the body's own natural repair mechanism.

### Tissue Preparation

In one embodiment, the inventive composition is seeded with precursor keratinocytes. As noted above, these cells can be isolated from a wide range of epithelial tissues including the basal epidermis, the hair bulge, the cornea limbus and neonatal foreskin.

Isolating precursor keratinocytes from the basal layer of the epidermis can be done using the split dermis technique as disclosed in U.S. Pat. No. 5,834,312 A and U.S. Pat. No. 7,037,721, the disclosures of which are incorporated herein by reference. In general, the split dermis technique begins by removing epidermal tissue using any suitable surgical technique, and subjecting the tissue to enzymatic digestion. Enzymes suitable for the digestion of the epithelial tissue include trypsin, chymotrypsin, collagenase, elastase, hyaluronidase, Dnase, pronase, and/or dispase. Following digestion, the dermal and epidermal layers are separated when the cornified side of the epidermis is placed on a clean sterile polystyrene surface whereupon the epidermis spontaneously detaches, and the dermis is removed with sterile forceps. Following separation of dermis from epidermis, the epidermis is dissociated into essentially single cells to form a suspension of epidermal cells in a liquid medium. Disassociation of the cells may be accomplished mechanically provided that shearing forces are avoided. Mechanical disassociation may be accomplished by stirring at low speeds, vortexing, pipetting, and other forms of mixing. and treatment of the epidermis with chelating agents that weaken the connections between neighboring cells.

Mechanical separation may be used to obtain a cell preparation with or without enzymatic digestion. Mechanical devices for this purpose include grinders, blenders, sieves, homogenizers, pressure cells, or insonators (Freshney, Culture of Animal Cells. A Manual of Basic Technique, 2d Ed., A. R. Liss, Inc., New York, 1987, Ch. 9, pp. 107-26; incorporated herein by reference).

Although isolation from the basal epidermis is specifically disclosed, one skilled in the art will appreciate that the precursor keratinocytes of the invention may be derived from any epithelial tissue including neonatal foreskin. Neonatal foreskin is a particularly good source of precursor keratinocytes because it is composed of up to 10% precursor keratinocytes (Toma, J. G. “Isolation and characterization of multipotent skin-derived precursors from human skin” 2005 June-July; 23(6):727-37).

Epithelial Stem Cell (Precursor Keratinocyte) Isolation

The precursor keratinocytes of the inventive composition may be isolated through a variety of techniques known in the art. Without limitation, these techniques include calcium stripping, fluorescence-activated cell-sorting (FACS) and collagen selection.

1. Calcium Stripping

The epithelial stem cells of the inventive composition are preferably isolated by calcium stripping. Calcium stripping is a process by which terminally differentiated keratinocytes are separated from the precursor keratinocytes of the basal epithelium. The procedure generally involves the culture of a mixed population of terminal keratinocytes and precursor keratinocytes in a calcium-free medium having less than 10-6 M calcium cations.

Calcium stripping as a means for isolating precursor keratinocytes is well documented in the art as demonstrated by the detailed procedures set out in U.S. Pat. No. 5,686,302, U.S. Pat. No. 5,834,312, U.S. Pat. No. 6,087,168, Hakkinen, L. “An improved method for culture of epidermal keratinocytes from newborn mouse skin” Methods Cell Sci. (2001) 23 (4): 189-196, Price, F. M. “Approaches to enhance proliferation of human epidermal keratinocytes in mass culture” J. Natl. Cancer Inst. (1983) 70(5):853-861; Babcock, M. S. “Clonal growth and serial propagation of rat esophageal epithelial cells” In Vitro (1983) 19(5):403-415, and Jensen, P. K. “Low Ca++ stripping of differentiation cell layers in human epidermal cultures: an in vitro model of epidermal regeneration” Exp. Cell Res. (1988) 175(1):63-73. The disclosures of these documents are incorporated herein by reference.

2. FACS

FACS is a procedure wherein ligand/signal conjugates are used to separate cells based on their cell-surface receptor profile. This method lends itself to the separation of precursor keratinocytes from other cells of the epidermis due to the differential expression of surface βi ntegrin. β integrins are heterodimeric glycoprotein adhesion receptors that secure precursor keratinocytes to the matrix proteins of the basement membrane. Because precursor keratinocytes express high levels of β integrin relative to other cells of the epidermis, FACS can be used to separate precursor keratinocytes from the remaining cells of the epidermis. Procedures for isolating precursor keratinocytes using FACS are detailed in U.S. Patent Application US20060073117 A1 and U.S. Pat. No. 6,485,971 B1, the disclosures of which are incorporated herein by reference.

3. Collagen Selection

Isolating precursor keratinocytes by collagen selection also involves the differential expression of β integrins. β integrins have a particular affinity for type IV collagen molecules. Thus, substrates coated with type IV collagen may be used to select precursor keratinocytes from a mixed population of cells. The procedure for isolating precursor keratinocytes is detailed in the article “Separation of Human Epidermal Stem (Cells from Transit Amplifying Cells on the Basis of Differences in Integrin Function and Expression” Cell 73:713-723 (1993), the disclosure of which is incorporated herein by reference.

Inoculating the Spongiform Scaffold

This invention relates to the inoculation/introduction of cells into a spongiform scaffold in order to make an inoculated spongiform scaffold free of mesenchymal cells which, upon transplantaton to the target site of an epithelial defect in a recipient, promotes the growth of cells or the generation of tissue at the target site.

Seeding is distinct from the spontaneous infiltration and migration of cells into a lattice from a wound site when the lattice is place at the wound site.

Accordingly, the spongiform scaffold is seeded with epithelial stem cells prior to implantation into a mammalian recipient. It should be understood that the seeded cells and their associated protein products direct migration of indigenous or native cells from neighboring tissue onto the scaffold and ultimately to replace the scaffold with native cells and tissue.

In one aspect of the invention, normal or non-disease state autologous host cells are harvested from an intended recipient and, expanded ex vivo to produce an inoculum of epithelial stem cells. The inoculum is then seeded onto the spongiform scaffold at an appropriate seeding density using a number of seeding techniques known in the art. Examples of seeding techniques for use with the invention include, but are not limited to, spreading, painting, spraying, soaking and pipetting. According to the invention, the spongiform scaffold is seeded with epithelial stem cells at a range of 100,000 to 1×106 cells per square centimer of scaffold. In Example 1 presented below, the spongiform scaffold was seeded with 550,000 cells per square centimer of scaffold. Regardless of the seeding density used, the inoculum and the scaffold of the inventive composition remain free of mesenchymal stem cells.

Spreading involves the use of an instrument such as a spatula to spread the inoculum across the spongiform scaffold. Seeding the scaffold by painting is accomplished by dipping a brush into the inoculum, withdrawing it, and wiping the inoculum-laden brush across the spongiform scaffold. This method suffers the disadvantage that substantial numbers of cells may cling to the brush, and not be applied to the lattice. However, it may nevertheless be useful, especially in situations where it is desired to carefully control the pattern or area of lattice over which the inoculum is distributed

Seeding the scaffold by spraying generally involves forcing the inoculum through any type of nozzle that transforms liquid into small airborne droplets. This embodiment is subject to two constraints. First, it must not subject the cells in solution to shearing forces or pressures that would damage or kill substantial numbers of cells. Second, it should not require that the cellular suspension be mixed with a propellant fluid that is toxic or detrimental to cells or woundbeds. A variety of nozzles that are commonly available satisfy both constraints. Such nozzles may be connected in any conventional way to a reservoir that contains an inoculum of epithelial stem cells.

Seeding the scaffold by pipetting is accomplished using pipettes, common “eye-droppers,” or other similar devices capable of placing small quantities of the inoculum on a collagen lattice. The aqueous liquid will permeate through the porous scaffold. The cells in suspension tend to become enmeshed in the scaffold, and are thereby retained upon or within the scaffold.

According to another embodiment of the invention, an inoculum of cells may be seeded by means of a hypodermic syringe equipped with a hollow needle or other conduit. A suspension of cells is administered into the cylinder of the syringe, and the needle is inserted into the spongiform scaffold. The plunger of the syringe is depressed to eject a quantity of solution out of the cylinder, through the needle, and into the scaffold. An important advantage of utilizing an aqueous suspension of cells is that it can be used to greatly expand the area of spongiform scaffold on which an effecitve inoculum is distributed. This provides two distinct advantages. First, if a very limited amount of intact tissue is available for autografting, then the various suspension methods may be used to dramatically increase the area or volume of a spongiform scaffold that may be seeded with the limited number of available cells. Second, if a given area or volume of a spongiform scaffold needs to be seeded with cells, then the amount of intact tissue that needs to be harvested from a donor site may be greatly reduced. The optimal seeding densities for specific applications may be determined through routine experimentation by persons skilled in the art.

The number and concentration of cells seeded into or onto a spongiform scaffold can be varied by modifying the concentration of cells in suspension, or by modifying the quantity of suspension that is distributed onto a given area or volume of spongiform scaffold.

The inoculated spongiform scaffold is then placed onto the target site of the subject's epithelial defect. Over time, the recipient's endogenous fibroblasts will regenerate, at the site of the epithelial defect, the connective tissue layer of the skin, while the transplanted precursor keratinocytes will regenerate the epithelial layer. Additionally, native cells integrate into the scaffold, any necessary vasculature develops, and the inoculated spongiform scaffold ultimately performs the function(s) of the tissue it was designed to replace or supplement. The spongiform scaffold, if formed of only biodegradable material, will be gradually reabsorbed as cell growth occurs, leaving in place an appropriately functioning replacement tissue.

### Skin Equivalent Assays

The inoculated spongiform scaffold of the invention in the parlance of transplantation is considered a “skin equivalent.” The skin equivalent of the invention is free of mesenchymal cells and is constructed by inoculating epithelial stem cells (e.g. precursor or progenitor keratinocytes) onto a spongiform scaffold.

Certain embodiments of the inventive spongiform scaffold relate to an in vitro, ex vivo or in vivo assay. Accordingly, the spongiform scaffold is used for determining the biological activity of pharmaceutical and/or biological agents, including, but not limited to cosmetics and electromagnetic/mechanical forces. This utility generally involves contacting a cell-inoculated spongiform scaffold with a test agent, and determining the biological activity the test agent has on the cells seeded on the scaffold. The test agent may be admninistered to a seeded scaffold in vitro, or it may be administered to the scaffold before and/or after the scaffold is transplanted into a recipient. In the environments noted, the biological effects of the test agent on the seeded cells, or cells that infiltrate the spongiform scaffold from the body of the recipient, may be measured. Biological effects measured with the inventive spongiform scaffold include, but are not limited to cytotoxicity, mutagenicity, proliferation, permeability, apoptosis, cell-to-cell interactions, gene regulation, protein expression, cell differentiation, cell migration and tissue formation. Test agents may be assessed individually, or as a combination of test agents.

The biological activity of a test agent may be measured using a variety of techniques known in the art. Cytoxicity, for example, may be measured using surrogate markers including, but not limited to, neutral red uptake, and lactate dehydrogenase release, and malondialdehyde levels (see e.g. Zhu et al. “Cytotoxicity of trichloroethylene and perchloroethylene on normal human epidermal keratinocytes and protective role of vitamin E” Toxicology April 1;209(1):55-67 Epub 2005 Jan. 7; and U.S. Pat. No. 5,891,161; these disclosures are incorporated herein by reference). Cytoxicity may also be measured by microscopically comparing the numbers of live cells before and after the spongiform scaffold is exposed to a test agent.

Cytotoxicity may be measured with the inventive composition by detecting the metabolic reduction of a soluble tetrazolium salt to a blue formazan precipitate since this reaction is dependent on the presence of viable cells with intact mitochondrial function. This assay is used to quantitate cytotoxicity in a variety of cell types, including cultured human keratinocytes (see e.g. U.S. Pat. No. 5,891,617 A, incorporated herein by reference). Other methods for measuring cytoxicity include examination of morphology, the expression or release of certain markers, receptors or enzymes, on DNA synthesis or repair, the measured release of [³H]-thymidine, the incorporation of BrdU, the exchange of sister chromatids as determined by by metaphase spread (see U.S. Pat. No. 7,041,438 B2 and “In vitro Methods in Pharmaceutical Research”, Academic Press, 1997; these are incorporated herein by reference), and the differential incorporation of specific dyes by viable and non-viable cells (see e.g. U.S. Pat. No. 6,529,835 B1, incorporated herein by reference).

Due to its incorporation of precursor keratinocytes, the inventive spongiform scaffold is particularly suited to evaluating skin toxicity and the efficacy of therapeutics aimed at treating the skin (see Hoh et al. “Multilayered keratinocyte culture used for in vitro toxicology” Mol. Toxicol. 1987-88 Fall; 1(4):537-46, incorporated herein by reference).

The inventive spongiform scaffold also provides methods of screening for agents that promote, inhibit or otherwise modulate the differentiation and/or proliferation of epithelial stem cells. There are a number of proliferation and differentiation assays known in the art including those disclosed in U.S. Pat. Nos. 7,037,719, 6,962,698, 6,884,589 and 6,824,973, the disclosures of which are incorporated herein by reference. In general, these assays involve culturing a population of progenitor cells in the presence of a test agent, and monitoring the proliferative and/or differentiating effects that the test agent imparts on the progenitor cell population., and on progenitor cell populations seeded on the inventive spongiform scaffold. One skilled in the art will appreciate that there are a number of methods for monitoring these effects including, but not limited to, testing for the presence of lineage-identifying cell surface markers, microscopic analysis of cell morphology, histological examination of extracellular proliferation markers, and cell counts.

### Spongiform Scaffold

Structure

The preferred spongiform materials of the invention are absorbable materials which are degraded in vivo and do not require removal from the target site. Particularly useful spongiform materials for use in the invention are hemostatic materials including, but not limited to, collagen, and oxidized cellulose.

Spongostan™ and Gelfoam™ have been available and used in various surgical procedures as a topical hemostatic agents since the mid 1940's. Spongostan is a brand of absorbable gelatin sterile sponge manufactured by Johnson and Johnson. It is a medical device intended for application to bleeding surfaces as a hemostatic. It is water insoluble, off-white, non-elastic, porous, pliable and prepared from purified porcine skin collagen. Spongostan can absorb and hold within its interstices, many times its weight in blood and other fluids. When not used in excessive amounts, Spongostan is completely absorbed with little tissue reaction. This absorption is dependent on several factors, including the amount used, degree of saturation with blood or other fluids, and the site of use. When placed on soft tissues Spongostan is usually absorbed completely in four to six weeks, without inducing excessive scar tissue. Becton Dickinson also manufactures spongiform scaffolds which provide a substrate for use with the invention for in vivo tissue regeneration.

Shaping/Manipulating Spongiform Scaffolds

The spongiform scaffold of the present invention may take on any configuration that permits the culture, implantation and/or grafting of the cells inoculated thereon. Such configurations include tubes, rolled and flat mats, fabrics, gauzes, hollow and solid cylinders, spheres, concave configurations, wedges, blocks, cubes and cones. For the spongiform scaffolds of the invention, thicknesses of sponges are suitably in the range of about 50 to 10,000 microns. In preferred embodiments, the spongiform scaffold is adapted to the shape of the epithelial defect in the recipient. Methods for shaping and manipulating a spongiform scaffold are disclosed in the following references: U.S. Pat. No. 2,610,625, U.S. Pat. No. 3,157,524, U.S. Pat. No. 3,368,911, U.S. Pat. No. 3,587,586, U.S. Pat. No. 4,215,693, U.S. Pat. No. 5,976,878, U.S. Pat. No. 6,365,149 B2, U.S. Pat. No. 6,986,735 B2, U.S. Pat. No. 6,835,336 B2 U.S. Pat. No. 6,572,650 B1, and U.S. Pat. No. 6,335,007 B1, the disclosures of which are incorporated herein by reference.

It is important to note that the shape of the spongiform scaffold will vary depending on the clinical requirements of the recipient's epithelial defect. For example, a method for treating hypospadia as disclosed herein relies on an inoculated spongiform scaffold that is in the shape of a tube. One skilled in the art will appreciate that this shape can be achieved by a number of techniques known in the art including manufacturing the spongiform scaffold as a continuous tube, by joining the edges of a planar spongiform scaffold to form a hollow cylinder, or wrapping a spongiform scaffold around a tube to form a reinforced, tubular spongiform scaffold.

In general, the surgeon exposes the defect or damaged area, if it is not naturally exposed as with an abrasion. A spongiform scaffold is sized and shaped sufficient to bridge, repair and/or reinforce the defect. The scaffold may be sutured in place as a temporary prosthesis. The spongiform scaffold is selected to be of a construction sufficient so that cells at the periphery or adjacent the subject's target tissue can grow into the scaffold and form a long-term biological tissue correction structure before the scaffold is completely bioabsorbed. The scaffold is then retained in position until the long-term biological tissue correction structure forms and the spongiform scaffold is completely bioabsorbed.

Scaffold Support Members

In one aspect of the invention, the scaffold is used in vivo as a prosthesis or implant to replace damaged or diseased tissue. The scaffold may be formed into an appropriate shape and then introduced or grafted into recipients such as a mammal, and in particular, a human recipient. The structure of the scaffold can be designed to mimic internal body structures (e.g. laryngeal and urethral), as well as external body structures.

Further modifications to the scaffold result in shapes and sizes that substantially fit the target site of an epithelial defect. Non-limiting examples of such spongiform scaffold shapes include sheets, tubes, cylinders, spheres, semi-circles, cubes, rectangles, wedges, and irregular shapes. Once the introduced scaffold is inoculated with cells, it serves as functional tissue.

In one aspect of the invention, the inoculated spongiform scaffold of the present invention may be used in conjunction with one or more support members that assist in providing support of the spongiform scaffold. Support members include, but are not limited to, catheters, tubes, stents, posts, hooks, bands and coils. These may be permanent or temporary structures as long as they are biocompatible. The inoculated, open celled polymeric spongiform scaffold matrix of the present invention may be formed around the support member (see example below for restoring a urethra). Alternatively, the spongiform scaffold may be formed, seeded with cells, and a support member added to the scaffolding prior to implantation into a recipient in need thereof. Additionally, the scaffold may be used in combination with other prostheses. For example, when used to replace or repair tubular organs, such as those in urogenital tract, larynx, and bile duct, it is helpful to use a stent. A stent is a generally longitudinal tubular device which is useful to open and support various lumens in the body. These devices are implanted within the vessel to open and/or reinforce collapsing or partially occluded sections of the vessel. In various embodiments, the spongiform scaffold may partially or fully coat or circumscribe the stent.

Spongiform Scaffold Attributes

The present invention is practiced with any material and shape thereof which (1) allows cells to attach to it (or can be modified to allow cells to attach to it); and (2) when implanted in a recipient, allows endogenous cells to migrate, penetrate, or otherwise occupy the spongiform scaffold thereby forming a new tissue.

Since the porous spongiform material contacts the wound bed, it should be non-immunogenic and possess certain other physical properties. It is, for instance, desirable to form the porous sponge from a material which initially wets and adheres to the wound bed. Close contact of the sponge with the wound surface confers a certain amount of stability to the biologically active wound dressing, thus preventing the movement of the graft relative to the wound surface. Close contact with the wound can be achieved by using pliable materials that effectively drape the wound. The porous, non-immunogenic, sponge layer should be insoluble in the presence of body fluids, but be slowly degradable in the presence of body enzymes. An exemplary material for this purpose is spongiform collagen. The sponge should have interconnected pores large enough for cell infiltration throughout the sponge.

A three dimensional scaffold desirably possesses sufficient mechanical strength to maintain its form when exposed to forces such as those exerted by cells in the scaffold's interior as well as pressure from surrounding tissue when implanted in situ.

Spongiform scaffolds may be formed from dried collagen foam, which incorporates the attributes of a solid, yet flexible, therapeutic device that can be cut or formed to the shape of a wound or lesion. The solid foam material is in a lightweight cellular form having gas, such as air, bubbles dispersed throughout. In this physical solid foam form, a dried hydrogel can be prepared with non-covalently bound materials “trapped” within its interstices such that the solid foam can serve as a device for delivering to a recipient cells, drugs, hemostatic agents or biological response modifier, and combinations thereof.

In order for a scaffold to perform properly, it must possess certain morphological and other characteristics. Among the most significant morphological characteristics of open celled materials are relative density and the correlative pore volume fraction, cell shape and uniformity, and to a lesser extent, cell size. Cells or pores are the void spaces within the material. Open celled materials mean the cells connect through open faces. In contrast, closed cell materials are made of cells that are closed off from one another.

In designing a material for use as a cellular scaffold, it is important for the pores to be of a sufficiently large size so as to allow cells (i.e., living cells) to maintain their shape within the structure. Additionally, an open cell configuration and a large pore volume fraction are desirable in order to allow a cell suspension to fully penetrate the structure and thus permit cell seeding and/or cell migration throughout the material. An insufficient pore size and/or pore volume fraction will restrict cells from gaining uniform access throughout the scaffold structure. Furthermore, free access of nutrients to the cells as well as efficient removal of waste products formed as a result of cellular metabolism will be impeded.

A method for making a porous foam is disclosed in U.S. Pat. No. 6,333,029 which is incorporated herein by reference. This foam finds use in tissue engineering, having a gradient architecture through one or more directions. The gradient is created by blending polymers to create a compositional gradient by timing the onset of a sublimation step in the freeze drying process used to form the foam. One or more growth factors may be incorporated into the structure.

The spongiform scaffold may be a porous woven or non-woven open-celled spongiform matrix scaffold having a substantially open architecture, which provides sufficient space for exogenous and endogenous cell infiltration while maintaining sufficient mechanical strength to withstand the contractile forces exerted by cells growing within the scaffold during integration of the scaffold into a target site within a host.

It is contemplated as within the invention to employ spongiform scaffolds made from polymers alone, as copolymers, or blends thereof. The polymers may be biodegradable, biostable, or combinations thereof.

Suitable natural polymers include polysaccharides such as alginate, cellulose, dextran, pullane, polyhyaluronic acid, chitin, poly(3-hydroxyalkanoate), poly(3-hydroxyoctanoate) and poly(3-hydroxyfatty acid). Also contemplated within the invention are chemical derivatives of said natural polymers including substitutions and/or additions of chemical groups such as alkyl, alkylene, hydroxylations, oxidations, as well as other modifications familiar to those skilled in the art. The natural polymers may also be selected from proteins such as collagen, zein, casein, gelatin, gluten and serum albumen.

Biodegradable synthetic polymers for use with the invention include poly alpha-hydroxy acids such as poly L-lactic acid (PLA), polyglycolic acid (PGA) and copolymers thereof (i.e., poly D,L-lactic co-glycolic acid (PLGA)), and hyaluronic acid. Poly alpha-hydroxy acids are particularly advantageous as they are approved by the FDA for human clinical use. It should be noted that certain polymers, including polysaccharides and hyaluronic acid, are water soluble. When using water soluble polymers it is important to render these polymers partially water insoluble by chemical modification, for example, by use of a cross linker.

In an embodiment which uses a cellulose-based matrix, an appropriate absorbable spongiform cellulose is regenerated oxidized cellulose sheet material, for example, Surgicel™ (Johnson & Johnson, New Brunswick, N.J.) which is available in the form of various sized strips or Oxycel® (Becton Dickinson, Franklin Lakes, N.J.) which is available in the form of various sized pads, pledgets and strips. The absorbable cellulose-based matrix can be combined with transplantable cells (e.g. basal keratinocyte cells) free of mesenchymal cells and, optionally, other active ingredients by soaking the absorbable sponge in a suspension of the cells, where the suspension liquid can have other bioactive ingredients dissolved therein.

In one embodiment of the invention, the spongiform scaffold is derived from purified bovine dermal collagen. Spongiform scaffolds of this embodiment are commercially available as Avitene™ (MedChem, Woburn, Mass.) which is available in various sizes of nonwoven web and fibrous foam, Helistat™ (Marion Merrell Dow, Kansas City, Mo.) which is available in various size sponges and Hemotene™ (Astra, Westborough, Mass.) which is available in powder form. The spongiform scaffold of the invention may also be derived from Porcine collagen. One commercially available porcine spongiform scaffold is Spongostan™ (Ethicon division of Johnson & Johnson).

As noted above, the absorbable collagen sponge may be derived from any source of biocompatible collagen. These include autologous, allogenic and xenogeneic sources of collagen, as well as collagen that is produced by recombinant DNA technology. Animal collagen for use with the inventive composition may be derived from humans, cows, pigs, sheep, goats, rabbits, mice, rats, horses or any other animal that serves as a reservoir of collagen that is biocompatible and supports the culture and/or implantation of the cells disclosed herein. Preferably, the inventive composition comprises porcine collagen due to its low antigenicity.

A collagen spongiform scaffold is prepared from any collagen rich animal tissue. One method for preparing the collagen sponge from beef tendon is disclosed in U.S. Pat. No. 2,610,625, the disclosure of which is incorporated by reference. Briefly, this method involves extracting colloidal collagen from beef tendon using acetic acid, freezing the colloidal collagen, and lyhophilizing the colloidal collagen to create a porous collagen sponge. Another method for preparing a collagen spongiform scaffold is disclosed in Japanese Unexamined Patent Publication No 43734/1993 which is incorporated herein by reference. This document teaches adding lipophilic organic solvent to a collagen solution, homogenizing said solution to expand, and then lyophilizing the homogenate. According to this method, spongiform scaffold having uniform pore size may be obtained. Another instructive reference includes U.S. Pat. No. 2,610,625 (incorporated herein by reference) which discloses tanning procedures which modify the sponge's resistance to breakdown by hydration and enzymatic digestion when the sponge is used in surgical applications.

The spongiform scaffold may be manufactured in the various shapes and sizes noted above. U.S. Pat. No. 3,157,524 discloses obtaining a desired shape for the spongiform scaffold by lyophilizing colloidal collagen in stainless steel form. This document, the disclosure of which is incorporated herein by reference, further teaches a method for making spongiform scaffolds in the shape of a tube. Briefly, this method involves freezing colloidal collagen around a supporting tube of desired diameter, and removing the tube after the colloidal collagen is lyophilized. The manufacture of tubular spongiform scaffolds made from collagen is also taught by U.S. Pat. No. 3,587,586, Doillon et al, J. Biomed. Materials Res., 20: 1219-1228 (1986) and R. C. Thompson, “Polymer Scaffold Processing,” in Principles of Tissue Engineering, Eds. R. Lanza et al., R. G. Landis Co. (1997), the disclosures of which are incorporated herein by reference

Making Spongiform Scaffolds

Spongiform scaffolds for use in the present invention are manufactured using techniques well known in the art (R. C. Thompson, “Polymer Scaffold Processing,” in Principles of Tissue Engineering, Eds. R. Lanza et al., R. G. Landis Co. (1997), incorporated herein by reference).

Scaffold morphology is directly related to the method and materials used to fabricate the structure. Spongiform scaffolds are known to be formed from natural or artificial polymers or combinations thereof. A variety of techniques are currently available for making tissue scaffolding and include fiber bonding, solvent casting and particulate leaching, membrane lamination, melt molding, polymeric/ceramic fiber composite foams, phase separation, and in situ polymerization. Depending on the raw materials and methods used, scaffolding can be made in a variety of shapes and sizes.

It is contemplated as within the invention to use the polymers alone, as copolymers, or blends thereof to fabricate spongiform scaffolds. Selection of the polymer combinations will depend upon the particular application and include consideration of such factors as desired tensile strength, elasticity, elongation, modulus, toughness, viscosity of the liquid polymer, whether biodegradable or permanent structures are intended, and the like to provide desired characteristics.

Polymers that degrade within one to twenty-four weeks are preferable. Synthetic polymers are preferred because their degradation rate can be more accurately determined and they have more lot to lot consistency and less immunogenicity than natural polymers. Natural polymers that can be used include proteins such as collagen, albumin, and fibrin; and polysaccharides such as alginate and polymers of hyaluronic acid. Synthetic polymers include both biodegradable and non-biodegradable polymers. Examples of biodegradable polymers include polymers of hydroxy acids such as polylactic acid (PLA), polyglycolic acid (PGA), and polylactic acid-glycolic acid (PLGA), polyorthoesters, polyanhydrides, polyphosphazenes, and combinations thereof. Non-biodegradable polymers include polyacrylates, polymethacrylates, ethylene vinyl acetate, and polyvinyl alcohols.

Polyanhydrides and polyvinyl chlorides are known to introduce flexibility into a polymer. It is possible, therefore, to use a small amount of certain polymers as additives to impart desired properties to the main polymer or polymer blend. For example, by adding some polyanhydride to a PLA polymer, flexibility of the structure formed thereof is increased. Small amounts of a non-biodegradable polymer may be added to a biodegradable polymer without compromising the biodegradability of the final material formed thereof. Selection of polymer blends, copolymers, and additives will be based on the particular end use of the polymeric matrix structure and can be made accordingly by one having ordinary skill in the art. It is therefore within the contemplation of the invention to employ multiple polymers, polymer blends, copolymers, and additives to maximize desirable spongiform scaffold properties.

Any material which is biocompatible and degrades at a suitable rate may be used. The pore volume fraction (PVF) is selected so as to encourage cellular penetration and growth throughout the scaffold. Generally a PVF of from 60>98% is desirable. Particularly advantageous is a PVF of greater than 80%. The pore volume fraction may be uniform or non-uniform.

When collagen is employed as biocompatible polymer, the spongiform scaffold may be degraded by the action of collagenase secreted by cells. However, resistance to collagenase may be imparted by introducing crosslinking to spongiform collagen sponge. An intensity of resistance thereof may be controlled by degree of crosslinking.

Introduction of crosslinking into said sponge of the invention may be carried out, for example, by heat-dehydration crosslinking (e.g. U.S. Pat. No. 6,039,760, U.S. Pat. No. 5,282,859, and RE 35,399, incorporated herein by reference), chemical crosslinking, etc. Crosslinking agents for chemical crosslinking include, but are not limited to glutaraldehyde, formaldehyde and like aldehydes; hexamethylene diisocyanate, tolylene diisocyanate, and like diisocyanates; ethyleneglycol diglycidylether, and like epoxides; and carbodiimide hydrochlorides etc., preferably include glutaraldehyde.

An advantage of using a biostable polymer in combination with a biodegradable polymer is that the biodegradable polymer can degrade over time allowing for full integration of cellular material in its place. The remaining biostable polymer portion may then remain and serve a support function to the newly integrated cellular material. Thus, this aspect of the invention is particularly beneficial for use with any organ in which mechanical strength of the tissue is important. The skilled artisan will appreciate that the spongiform scaffold may be reinforced with non-biodegradable, non-polymeric supports including, but not limited to, biocompatible alloys and nylons.

Among natural polymers that can be easily formed into a porous spongy matrix, there is a particular interest in chitosan. Chitosan is a linear polysaccharide obtained from partial deacetylation of chitin that can be derived from arthropod exoskeletons. Chitin is slowly degraded in vivo and thus, chitin and its degradation products are natural and safe. In the pharmaceutical field, chitosan has been used as a vehicle for the sustained release of drugs (Hou et al., Chem Pharm Bull 1985; 33(9):3986-3992). Chitin as such has been woven into fabrics and used as dressings for wound healing.

One of ordinary skill in the art would refer to the following references for guidance in making spongiform scaffolds suitable for use in the invention: Kemnitzer and Kohn, in the Handbook of Biodegradable Polymers, edited by Domb, Kost and Wisemen, Hardwood Academic Press, 1997, pages 251-272. Copoly(ether-esters) for the purpose of this invention include those copolyester-ethers described in “Journal of Biomaterials Research”, Vol. 22, pages 993-1009, 1988 by Cohn and Younes and Cohn, Polymer Preprints (ACS Division of Polymer Chemistry) Vol. 30(1), page 498, 1989 (e.g. PEO/PLA); Allcock in The Encyclopedia of Polymer Science, Vol. 13, pages 31-41, Wiley Intersciences, John Wiley & Sons, 1988 and by Vandorpe, Schacht, Dejardin and Lemmouchi in the Handbook of Biodegradable Polymers, edited by Domb, Kost and Wisemen, Hardwood Academic Press, 1997, pages 161-182. Polyorthoesters such as those described by Heller in Handbook of Biodegradable Polymers, edited by Domb, Kost and Wisemen, Hardwood Academic Press, 1997, pages 99-118 (hereby incorporated herein by reference).

### Growth Medium and Cofactors

The embryonic stem cells of the invention may be grown in complex or simple media. Furthermore, although the cultures may be grown in a media containing sera or other biological extracts, neither serum nor any other biological extract is required. Moreover, the cell cultures can be maintained in the absence of serum for extended periods of time.

The point regarding growth in minimal media is important. At present, most media or systems for prolonged growth of mammalian cells incorporate undefined proteins or use feeder cells to provide proteins necessary to sustain such growth. Because the presence of such undefined proteins can interfere with the intended end use of the subject culture, it will generally be desirable to culture the cells under conditions to minimize the presence of undefined proteins.

As used herein the language “minimal medium” refers to a chemically defined medium which includes only the nutrients that are required by the cells to survive and proliferate in culture. Typically, minimal medium is free of biological extracts, e.g., growth factors, serum, or other substances which are not necessary to support the survival and proliferation of a cell population in culture. For example, minimal medium generally includes at least one amino acid, at least one vitamin, at least one salt, at least one antibiotic, at least one indicator, e.g., phenol red (used to determine hydrogen ion concentration), glucose, and other miscellaneous components necessary for the survival and proliferation of the cells. Minimal medium is serum-free. A variety of minimal media are commercially available from Gibco BRL, Gathersburg, Md., as minimal essential media.

Growth factors for use with the inoculated spongiform scaffold may be introduced through the genetic modification of epithelial stem cells. According to this embodiment, epithelial stem cells are transfected with exogenous, growth factor-encoding polynucleotides. Techniques for transfecting epithelial stem cells are known in the art and include transfection by recombinant viruses (see e.g. U.S. Pat. Nos. 6,969,608 and 6,927,060, Kolodka, T. M. “Evidence for keratinocyte stem cells in vitro: long term engraftment and persistence of transgene expression from retrovirus-transduced keratinocytes” PNAS April 14;95(8):4356-61, 1998, Fenves, E. S., “Approaches to gene transfer in keratinocytes,” J. Invest. Dermatol. 103(5):70S-75S, and Garlick, J. A., “Retrovirus-mediated transduction of cultured epidermal keratinocytes,” J. Invest. Dernatol. 97:824-829, 1991, incorporated herein by reference), lipofectamine transfection (U.S. Pat. No. 6,969,608, incorporated herein by reference), and polycationic lipid transfection (U.S. Pat. No. 6,884,595, incorporated herein by reference). One skilled in the art will appreciate that the epithelial stem cells of the spongiform scaffold may be transfected using any suitable technique that introduces exogenous polynucleotide(s) while maintaining the stem cell's regenerative capabilities. Such techniques include, without limitation, electroporation and calcium precipitation.

However, while growth factors and regulatory factors need not be added to the media, the addition of such factors, or the inoculation of other specialized cells may be used to enhance, alter or modulate proliferation and cell maturation in culture. The growth and activity of cells in culture can be affected by a variety of growth factors such as insulin, growth hormone, somatomedins, colony stimulating factors, erythropoietin, epidermal growth factor, and hepatic erythropoietic factor (hepatopoietin. Other factors which regulate proliferation and/or differentiation include prostaglandins, interleukins, and naturally-occurring negative growth factors, fibroblast growth factors, and members of the transforming growth factor β family.

Certain biologically active agents are useful in improving the performance of three dimensional scaffolds. For example, extracellular matrix (ECM) molecules consisting of secreted proteins and polysaccharides occupy the intercellular space and bind cells and tissues together. Cells can attach to matrix proteins by interacting with them through cell adhesion molecules such as integrins. It is believed that the presence of ECM molecules in a three dimensional scaffold may act to improve cell adhesion. In addition, the presence of signaling and ECM molecules can encourage cells to perform their differentiated tissue specific functions. These properties can facilitate the scaffold to serve its function as either a living tissue equivalent or as a model tissue system.

It is further within the contemplation of the present invention to add tissue specific ECM proteins to the spongiform scaffold. Appropriate ECM proteins may be added to the scaffold in order to further promote cell ingrowth, tissue development, and cell differentiation within the scaffold. Alternatively, the scaffold of the present invention can include ECM macromolecules in particulate form or include extracellular matrix molecules deposited by viable cells.

Extracellular matrix molecules for use with the inventions are commercially available. For example, extracellular matrix from EHS mouse sarcoma tumor is available. (Matrigel™, Becton Dickinson, Corp. Medford, Mass). Examples of ECM proteins for use with the invention include, but are not limited to, fibronectin, laminin, vitronectin, tenascin, entactin, thrombospondin, elastin, gelatin, collagen, fibrillin, merosin, anchorin, chondronectin, link protein, bone sialoprotein, osteocalcin, osteopontin, epinectin, hyaluronectin, undulin, epiligrin, and kalinin. Other extracellular matrix molecules are described in Kleinman et al., J. Biometer. Sci. Polymer Edn., 5: 1-11, (1993), herein incorporated by reference. It is intended that the term encompass presently unknown extracellular matrix proteins that may be discovered in the future, since their characterization as an extracellular matrix protein will be readily determinable by persons skilled in the art. The ECM proteins described herein may be used alone or in combination in manufacturing the spongiform scaffold.

Additional biologically active macromolecules helpful for cell growth, morphogenesis, differentiation, and tissue building include growth factors, proteoglycans, glycosaminoglycans and polysaccharides. These compounds are believed to contain biological, physiological, and structural information for development and/or regeneration of tissue structure and function. These compounds are described in the literature and are also commercially available.

Growth factors for use with the invention can be prepared using methods known to those of skill in the art. For example, growth factors can be isolated from tissue, produced by recombinant means in bacteria, yeast or mammalian cells. EGF can be isolated from the submaxillary glands of mice. Genetech (San Francisco, Calif.) produces TGF-β recombinantly. Many growth factors are also available commercially from vendors including: Sigma Chemical Co., St. Louis, Mo.; Collaborative Research, Los Altos, Calif.; Genzyme, Cambridge, Mass.; Boehringer, Germany; R&D Systems, Minneapolis, Minn.; and GIBCO, Grand Island, N.Y. The commercially available growth factors may be obtained in both natural and recombinant forms.

The term “growth factors” is art recognized and is intended to include, but is not limited to, one or more of platelet derived growth factors (PDGF), e.g., PDGF AA, PDGF BB; insulin-like growth factors (IGF), e.g., IGF-I, IGF-II; fibroblast growth factors (FGF), e.g., acidic FGF, basic FGF, β endothelial cell growth factor, FGF 4, FGF 5, FGF 6, FGF 7, FGF 8, and FGF 9; transforming growth factors (TGF), e.g., TGF-P1, TGF-β 1.2, TGF-β 2, TGF-β 3, TGF-β 5; bone morphogenic proteins (BMP), e.g., BMP 1, BMP 2, BMP 3, BMP 4; vascular endothelial growth factors (VEGF), e.g., VEGF, placenta growth factor; epidermal growth factors (EGF), e.g., EGF, amphiregulin, β-cellulin, heparin binding EGF; interleukins, e.g., IL-1, IL-2, IL-3, IL-4, IL-5, IL-6, IL-7, IL-8, IL-9, IL-10, IL-11, IL-12, IL-13, IL-14; colony stimulating factors (CSF), e.g., CSF-G, CSF-GM, CSF-M; nerve growth factor (NGF); stem cell factor; hepatocyte growth factor, and ciliary neurotrophic factor. Additional growth factors are described in Sporn and Roberts, Peptide Growth Factors and Their Receptors I, Springer-Verlag, New York (1990) which is hereby incorporated by reference. It is intended for the term “growth factors” to encompass presently unknown growth factors that may be discovered in the future, since their characterization as a growth factor will be readily determinable by persons skilled in the art.

Other biologically active agents such as nutrients, cytokines, hormones, angiogenic factors, immunomodulatory factors, and drugs are also expected to aid the cells in thriving in the scaffold matrix. As a result, it is therefore within the scope of the present invention to include one or more of these useful compounds within the scaffold to further promote cell ingrowth and tissue development and organization within the scaffold. These are described in the literature and are also commercially available.

Furthermore, biologically active short peptide sequences derived from proteins may also be used. For example, cell adhesion may be enhanced by a number of short peptide sequences derived from adhesion proteins. These sequences are able to bind to cell-surface receptors and mediate cell adhesion with an affinity similar to that obtained with intact proteins. Arg-Gly-Asp (RGD) is one such peptide which may be coated onto the surfaces of three dimensional scaffolds to increase cell adhesion. This sequence binds to integrin receptors on a wide variety of cell types.

The term “proteoglycan” is art recognized and is intended to include one or more of decorin and dermatan sulfate proteoglycans, keratin or keratan sulfate proteoglycans, aggrecan or chondroitin sulfate proteoglycans, heparan sulfate proteoglycans, biglycan, syndecan, perlecan, or serglycin.

The term “proteoglycans” encompasses presently unknown proteoglycans that may be discovered in the future, since their characterization as a proteoglycan will be readily determinable by persons skilled in the art. The term “glycosaminoglycan” is art recognized and is intended to include one or more of heparan sulfate, chondroitin sulfate, dermatan sulfate, keratan sulfate, hyaluronic acid. The term encompasses presently unknown glycosaminoglycans that may be discovered in the future, since their characterization as a glycosaminoglycan will be readily determinable by persons skilled in the art.

The term “polysaccharide” is art recognized and is intended to include one or more of heparin, dextran sulfate, chitin, alginic acid, pectin, and xylan. The term encompasses presently unknown polysaccharides that may be discovered in the future, since their characterization as a polysaccharide will be readily determinable by persons skilled in the art.

### EXAMPLES

### Example 1

Obtaining Epithelial Stem Cells—Progenitor Keratinocytes

Skin Biopsy and Enrichment of Progenitor Keratinocytes

1. Immediately after biopsy, the skin segment was placed in the container with transport medium (RPMI—5% Fetal Bovine Serum).

2. The sample container was sprayed with 70% ethyl alcohol and placed in a hood located in a tissue culture room.

3. The transport medium was removed by a 100 ml pipette.

4. The skin segment was placed into the sterile 250 ml empty bottle.

5. 100 ml of Tobramycin-PBS solution (160 mcg/ml final concentration) was added into the bottle by a 100 ml pipette.

6. The bottle was gently rocked.

7. The Tobramycin-PBS solution was decanted by a 100 ml pipette.

8. The pipettes were changed between washings.

9. Steps 5-9 were repeated for a total 10 times.

10. The specimen was transferred onto the sterile flax pad using 8 inch forceps.

11. The fat was removed using a sterile scalpel.

12. The remaining strip was cut into pieces approximately 3 mm wide using a sterile scalpel.

13. The obtained pieces were placed into a 50 ml plastic tube.

14. 30 ml of the Tobramycin-PBS solution was added to the tube by a 100 ml pipette.

15. The obtained pieces were washed 2 times in Tobramycin-PBS solution.

16. The skin pieces were transferred into a 50 ml plastic tube using forceps.

17. 10 ml of the cold 0.125% Dispase-DMEM solution was added to the tube.

18. The tube with skin pieces in Dispase-DMEM solution was incubated at 4° C. for 18 hr in a refrigerator.

19. The skin pieces were transferred into the Petri dish using forceps.

20. The epidermis was peeled off using wide-ended forceps along the basal plate.

21. The pieces of epidermis were placed into the cover of the Petri dish.

22. The pieces of epidermis were transferred into a 50 ml plastic tube.

23. 5 ml of the 0.125% Trypsin—0.5 mM EDTA solution was added to the tube by a sterile 10 ml pipette.

24. The tube was placed in the water bath and incubated for 1-5 min at 37° C. periodically shaking it until the pieces were dissolved.

25. 5 ml of transport medium (see step 1) was added to the tube to inhibit trypsin.

26. The mixture was pipetted a few times to obtain a single cell suspension of keratinocytes by a sterile 10 ml pipette.

27. The cell suspension was filtered through the 200 μ mesh into the 50 ml plastic tube.

28. The filtered cell suspension was centrifuged at 1000 rpm for 10 min.

29. The pellet was resuspended in 5 ml of keratinocyte culture medium containing DMEM/F12, 10% FBS, 10 ng/ml EGF, 5 mcg/rnl Insulin, 10⁻⁶ M Isopretonolol. Alternatively, the cells were cultured in a Progenitor Cell Targeted (PCT) Epidermal Keratinocyte medium (Chemicon) specially formulated to maintain growth of undifferentiated keratinocytes. In this case, the stripping procedure (Step 34) was omitted.

30. The cell count was determined using hematocytometer.

31. The suspension of keratinocytes was seeded into Collagen I coated flasks in keratinocytes culture medium (seeding concentration 2×10⁵ /ml).

32. The flasks were placed in 5% CO2 incubator and incubated for 10-14 days.

33. The medium was changed every other day until confluency and every day afterwards.

34. All differentiated cells were stripped off by incubating cultures in Ca2+− free DMEM for 24-48 hr.

35. The adherent keratinocytes were harvested by 0.25% Trypsin-EDTA and frozen in liquid nitrogen.

36. An aliquot of cells was submitted for testing for bacteria, mycoplasma, and endotoxin.

Spongiform Scaffold Preparation

37. The Spongostan film pack (J&J) was opened under the biosafety hood.

38. The 6 cm sponge was cut using a sterile scissors and placed into a sterile 6 cm Petri dish.

39. The 1% Collagen I solution in 0.1% acetic acid was poured into the Petri dish and placed into 37° C. thermostat for 20 min.

40. The sponge was washed in Hank's balanced salt solution 3-4 times in the Petri dish under the biosafety hood.

41. The keratinocyte culture medium or PCT medium (see step 29) was added to the washed film, the film was incubated at 37° C. for 4-6 hr and submitted for skin equivalent preparation.

Seeding the Spongiform Scaffold

42. The Collagen-coated sponge was placed into a 6 cm sterile Petri dish.

43. The previousely prepared frozen keratinocytes (see step 35), which passed sterility, mycoplasma, and endotoxin tests were thawed and the cell count was determined in hematocytometer.

44. The cell suspension was seeded at the density 5.5×10⁵cells/cm² of collagen I coated Spongostan (see step 40) in keratinocyte culture medium. The cell suspension was alternatively seeded using PCT medium (see step 29) and incubated in 5% CO2 incubator for 3-4 days.

45. In the case of keratinocyte medium, during last 24 hr the cells were incubated under serum-free conditions.

### Example 2

Transplantation—Hypospadia

The inventive procedure was performed on patients between the ages of about 1 to 6 years old. The physical characteristics of the patiens included some or all of the following: splitting of the foreskin along the ventral surface; splitting along the scrotum; actopic meatus in the proximal part of scrotum; significant ventral deformation of corpora cavernosa; splitting along the ventral surface of the prepuce; urethral opening of about #8CH in size; distortion of the penis toward the scrotum; dysplasia of the ventral penis; a hypospadias meatus located in the proximal part of the split scrotum; and the inability to direct a urine stream.

Surgical Procedure

The surgery began by making a circumferential cut around the penis glans, and extending the cut longitudinally along the ventral surface of the penis to the hypospadias meatus. The skin was immobilized until the penis basement and fibrous chordee which deforms the penis was excised, at which point the patient was ready to receive the transplant. Meanwhile, a wrapped spongiform scaffold was prepared from the seeded Spongostan scaffold from step 44 above. This was done by wrapping the seeded scaffold around a polyvinyl pediatric urethral catheter with a tube diameter of between 3-5 mm. The length of the catheter was determined by the distance between the subject's defective urethral opening, and the desired location of the urethral opening (e.g. the tip of the penis).

On the dorsal surface of the penis, a rectangle skin segment on the blood vessel peduncle was excised and formed around a urethral catheter #8 Ch. The proximal part of the skin wound on the dorsal surface of the penis orifice was formed by parting tissue, equal in size to the diameter of the penis, which was moved via the formed orifice. Then the urethral anastomosis between its proximal end distal end of the transplant (from the end to the end) on the catheter was created, then the distal part of the formed urethra was sutured to the top of the penis glans. The part of the foreskin, which is not involved in the plastic surgery, was moved from dorsal surface to the level of the glans. Epidermis from this part of the foreskin (prepuce) was removed. After this the erectile tissue of the penis glans along lateral and ventral surfaces was mobilized, lateral margins of glans were connected by stitches above the distal end of the artificial urethra and the wound was filled in by local tissue and sutured. The urethral catheter # 8 Ch was connected by surgical stitches to the skin of the penis glans by thread PDS 5/0. The placement of a bandage with glycerin completed the surgery. The progress of the transplant was monitored and the urethral removed at an average of 10 days after the surgery.

Results

The subjects were examined 6 months after surgery. Each subject's penis developed according the patient's age. Erections did not show deformation of the corpora cavernosa. The size of urethra was an average of #11 CH. Patients were able to direct the urinary stream. In eight operations performed on 5 children using the spongiform scaffold of the invention seeded with keratinocyte precursor cells free of mesenchyme, the success rate was 90%. Clinical and histological appearance of the above grafts in the eight operations of the RDEB children suggested that there was no rejection.

We claim:

1. A spongiform scaffold comprising epithelial stem cells, wherein said spongiform scaffold is free of mesenchymal cells.

2. The scaffold of claim 1, wherein said epithelial stem cells comprise one or more epithelial stem cell lines.

3. The scaffold of claim 1, wherein said epithelial stem cells are either autologous, allogeneic, xenogeneic or mixtures thereof in relation to a recipient.

4. The scaffold of claim 1, wherein said epithelial stem cells are precursor keratinocytes.

5. The scaffold of claim 1, wherein said epithelial stem cells are inoculated at a density sufficient to correct an epithelial defect.

6. The scaffold of claim 1, wherein said scaffold is adapted to a shape of a target site.

7. The scaffold of claim 1, wherein said scaffold has a shape selected from the group consisting of a planar shape, a three-dimensional shape, and combinations thereof.

8. The scaffold of claim 7, wherein said planar shape is selected from the group of shapes consisting of substantially circular, semi-circular, oval, irregular, rectilinear, and combinations thereof.

9. The scaffold of claim 7, wherein said three-dimensional shape is selected from the group consisting of a tube, a cylinder, a sphere, a cube, a wedge, and combinations thereof.

10. The scaffold of claim 8 or 9, wherein said scaffold is configured to substantially fit a target site.

11. The scaffold of claim 7, further comprising a support structure.

12. The scaffold of claim 11, wherein said support structure is a tube.

13. The scaffold of claim 12, wherein said tube has an interior wall, an exterior wall, and a wall thickness, said wall thickness being from about 50 microns, to about 10,000 microns.

14. The scaffold of claim 1, wherein said scaffold comprises a pore size that is sufficient to accommodate a diameter of an epithelial cell in at least a portion of said scaffold.

15. The scaffold of claim 14, wherein said scaffold, when implanted in a recipient, permits the growth of said epithelial stem cells and the ingrowth of cells from the body of said recipient.

16. The scaffold of claim 1, wherein said scaffold further comprises a non-biodegradable supporting structure.

17. The scaffold of claim 1, wherein said scaffold is a biodegradable polymer selected from the group consisting of a synthetic polymer, a natural polymer, and combinations thereof.

18. The scaffold of claim 17, wherein said biodegradable polymer comprises at least one of poly L-lactic acid (PLA), polyglycolic acid (PGA), alginate, collagen, hyaluronic acid, copolymers and blends thereof.

19. The scaffold of claim 17, wherein said biodegradable polymer comprises alginate or collagen.

20. The scaffold of claim 17, wherein said biodegradable polymer comprises collagen, and wherein said scaffold comprises Spongostan.

21. The scaffold of claim 1, wherein said scaffold further comprises at least one signal for modifying cell adhesion, cell growth, cell differentiation and/or cell migration, and wherein said at least one signal is added exogenously to said scaffold, is expressed by epithelial stem cells which have been genetically modified with at least one polynucleotide encoding said at least one signal, or combinations thereof.

22. The scaffold of claim 21, wherein said at least one signal comprises at least one biologically active agent selected from the group consisting of a nutrient, an angiogenic factor, an immunomodulatory factor, a drug, a cytokine, an extracellular protein, a proteoglycan, a glycosaminoglycan, a polysaccharide, a growth factor, an Arg-Gly-Asp (RGD) peptide, and modifications thereof.

23. The scaffold of claim 22, wherein said extracellular protein is at least one of a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, a kalinin, and modifications thereof.

24. The scaffold of claim 22, wherein said growth factor is at least one of a platelet-derived growth factor, an insulin-like growth factor, a fibroblast growth factor, a transforming growth factor, a bone morphogenic protein, a vascular endothelial growth factor, a placenta growth factor, an epidermal growth factor, an interleukin, a colony stimulating factor, a nerve growth factor, a stem cell factor, a hepatocyte growth factor, a ciliary neurotrophic factor, and modifications thereof.

25. The scaffold of claim 1, wherein at least a portion of said epithelial stem cells are genetically altered.

26. A method for generating tissue in a subject, the method comprising delivering an epithelial stem cell-inoculated spongiform scaffold free of mesenchymal cells to a target site comprising an epithelial defect in said subject, wherein said delivering allows said epithelial stem cells inoculated on said spongiform scaffold to differentiate thereby producing epithelial tissue at said target site.

27. The method of claim 26, wherein said epithelial stem cells comprise one or more epithelial stem cell lines.

28. The method of claim 26, wherein said epithelial stem cells are either autologous, allogeneic, xenogeneic or mixtures thereof in relation to said subject.

29. The method of claim 26, wherein said epithelial stem cells are precursor keratinocytes.

30. The method of claim 26, wherein said epithelial stem cells are inoculated at a density sufficient to correct an epithelial defect.

31. The method of claim 26, wherein said scaffold is adapted to a shape of said target site.

32. The method of claim 26, wherein said scaffold has a shape selected from the group consisting of a planar shape, a three-dimensional shape, and combinations thereof.

33. The method of claim 32, wherein said planar shape is selected from the group of shapes consisting of substantially circular, semi-circular, oval, irregular, rectilinear, and combinations thereof.

34. The method of claim 32, wherein said three-dimensional shape is selected from the group consisting of a tube, a cylinder, a sphere, a cube, a wedge, and combinations thereof.

35. The method of claim 33 or 34, wherein said scaffold is configured to substantially fit said target site.

36. The method of claim 32, wherein said scaffold further comprises a support structure.

37. The method of claim 36, wherein said support structure is a tube.

38. The method of claim 37, wherein said tube has an interior wall, an exterior wall, and a wall thickness, said wall thickness being from about 50 microns, to about 10,000 microns.

39. The method of claim 26, wherein said scaffold has a pore size that is sufficient to accommodate the ingrowth of cells from the body of said subject.

40. The method of claim 39, wherein said scaffold, when implanted in said subject, permits the growth of said epithelial stem cells and the ingrowth of cells from the body of said subject.

41. The method of claim 26, wherein said scaffold further comprises a non-biodegradable supporting structure.

42. The method of claim 26, wherein said scaffold is a biodegradable polymer selected from the group consisting of a synthetic polymer, a natural polymer, and combinations thereof.

43. The method of claim 42, wherein said biodegradable polymer is at least one of a poly L-lactic acid (PLA), polyglycolic acid (PGA), alginate, collagen, hyaluronic acid, copolymers and blends thereof.

44. The method of claim 42, wherein said biodegradable polymer comprises alginate or collagen.

45. The method of claim 43, wherein said biodegradable polymer comprises collagen, and wherein said scaffold comprises Spongostan.

46. The method of claim 26, wherein said scaffold further comprises at least one signal for modifying cell adhesion, cell growth, cell differentiation, and/or cell migration, and wherein said at least one signal is added exogenously to said scaffold, is expressed by epithelial stem cells which have been genetically modified with at least one polynucleotide encoding said at least one signal, or combinations thereof.

47. The method of claim 46, wherein said at least one signal comprises at least one biologically active agent selected from the group consisting of a nutrient, an angiogenic factor, an immunomodulatory factor, a drug, a cytokine, an extracellular protein, a proteoglycan, a glycosaminoglycan, a polysaccharide, a growth factor, a RGD peptide, and modifications thereof.

48. The method of claim 47, wherein said extracellular protein is at least one of a fibronectin, a laminin, a vitronectin, a tenascin, an entactin, a thrombospondin, an elastin, a gelatin, a collagen, a fibrillin, a merosin, an anchorin, a chondronectin, a link protein, a bone sialoprotein, an osteocalcin, an osteopontin, an epinectin, a hyaluronectin, an undulin, an epiligrin, a kalinin, and modifications thereof.

49. The method of claim 47, wherein said growth factor is at least one of a platelet-derived growth factor, an insulin-like growth factor, fibroblast growth factor I, fibroblast growth factor II, a transforming growth factor, a bone morphogenic protein, a vascular endothelial growth factor, a placenta growth factor, an epidermal growth factor, an interleukin, a colony stimulating factor, a nerve growth factor, a stem cell factor, a hepatocyte growth factor, a ciliary neurotrophic factor, and modifications thereof.

50. The method of claim 26, wherein at least a portion of said epithelial stem cells are genetically altered.

51. A method of making an inoculated spongiform scaffold for treating an epithelial defect in a recipient, the method comprising the steps of:
a. providing an inoculum of epithelial stem cells free from mesenchymal cells; and
b. inoculating a spongiform scaffold with a sufficient number of said epithelial stem cells in said inoculum to restore the epithelium at said epithelial defect,
wherein said scaffold remains free of mesenchymal stem cells prior to implantation in said recipient.

52. The method of claim 51, wherein said epithelial stem cells comprise one or more epithelial stem cell lines.

53. The method of claim 51, wherein said epithelial stem cells are precursor keratinocytes.

54. A method for regenerating tissue in a recipient having an epithelial defect, said method comprising the step of implanting a tissue-forming structure in said recipient, said a tissue-forming structure comprising an epithelial stem cell-inoculated spongiform scaffold, said scaffold being free of mesenchymal cells.

55. The method according to claim 54, wherein said epithelial defect is a skin defect or a urological defect.

56. The method of claim 55, wherein said urological defect is hypospadias, the method further comprising wrapping said scaffold around a tubular stent to form a scaffold-wrapped stent, and implanting said scaffold-wrapped stent into the penis of said recipient.

57. The method of claim 56, wherein said scaffold-wrapped stent is implanted in the corpora cavernosa.

58. A method of promoting tissue generation at a site of an epithelial defect in a subject, said method comprising the steps of:
a. inoculating a spongiform scaffold with epithelial stem cells, wherein said inoculated spongiform scaffold is free of mesenchymal cells; and
b. placing said inoculated spongiform scaffold in contact with said defect for a sufficient period of time to permit new epithelial tissue to develop at said site.

59. The method of claim 58, wherein said inoculated spongiform scaffold supports the differentiation of epithelial cells into a cell lineage to an extent sufficient to generate tissue said new epithelial tissue, and sufficient time is allowed to elapse for mesenchymal cells from said site to infiltrate into said spongiform scaffold.

60. A method for correcting hypospadias in a male patient, said method comprising the step of placing into the corpora cavemosa of said male patient an epithelial stem cell-inoculated spongiform scaffold, wherein in said spongiform scaffold is free of mesenchymal cells.

61. The method of claim 60, wherein the placing of said epithelial stem cell-inoculated spongiform scaffold allows for the growth of said epithelial stem cells and for the ingrowth of surrounding tissue cells into said scaffold, and wherein said growth elongates the patient's urethra toward the distal end of the penis.

62. The method of claim 60, wherein said epithelial stem cells are selected from the group consisting of autologous cells, allogeneic cells, xenogeneic cells, and combinations thereof.

63. The method of claim 62, wherein said epithelial stem cells are obtained from a cell bank.

64. A method for reconstructing a urethra in a patient, comprising the steps of
a. providing an inoculated spongiform scaffold, wherein said scaffold is inoculated with epithelial stem cells and is free of mesenchymal cells;
b. positioning said scaffold around a tubular support to form a supported scaffold; and
c. implanting said supported scaffold into the penis of said patient, whereby a reconstructed urethra is formed.

65. The method of claim 64, wherein said supported scaffold is implanted in the corpora cavernosa of said patient.

66. A method for testing the biological activity of an agent comprising
a. contacting said agent with a spongiform scaffold comprising epithelial stem cells, wherein said spongiform scaffold is free of mesenchymal cells; and
b. determining the effect of said agent on said epithelial stem cells.

67. The method of claim 66, wherein said determining step measures at least one of cell growth, cell death, cell differentiation and cell-to-cell interactions.

68. The method of claim 66, wherein said agent is at least one of a protein, a small molecule, a polysaccharide, a nucleotide, a polynucleotide, an amino acid, and an oligosaccharide.

69. The method of claim 66, wherein said agent comprises physical or electromagnetic energy.

70. The method of claim 66, wherein said determining step provides an indication of at least one of cytotoxicity, mutagenicity, proliferation, permeability, apoptosis, gene regulation, protein expression, and differentiation.

71. The method of claim 66, wherein said determining step provides an indication of the biological activity said agent will have on the skin of an animal.
